(19) United States
(12) Plant Patent Application Publication
(10) Pub. No.: US 2006/0059594 P1
(43) Pub. Date: Mar. 16, 2006

(54) Heuchera plant named 'Chinook'

(50) HEUCHERA MICRANTHA X HEUCHERA SANGUINEA; HEUCHERA

(75) Inventor: Harini Korlipara, Canby, OR (US)

Correspondence Address: KLARQUIST SPARKMAN, LLP, 121 SW SALMON STREET, SUITE 1600, PORTLAND, OR, 97204, US

(73) Assignee: Terra Nova Nurseries, Inc.

(21) Appl. No.: 10/942,361

(22) Filed: Sep. 15, 2004

(51) Int. Cl. A01H 5/00

(52) U.S. Cl. PLT/263

(57) ABSTRACT

A new and distinct hybrid of Heuchera plant characterized by its large coral colored flowers on short stalks, dark slightly ruffled foliage, and a compact habit.

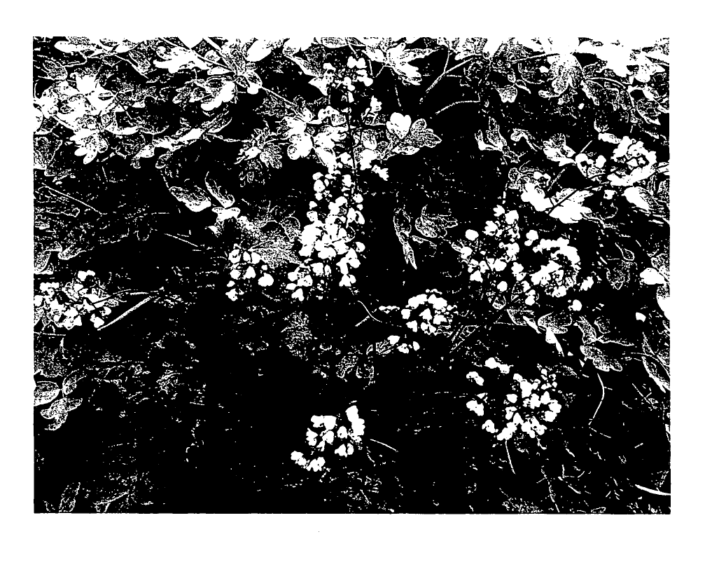

### BOTANICAL CLASSIFICATION

Heuchera (H. micrantha×H. sanguinea) hybrid

### VARIETY DENOMINATION

‘Chinook’

The present invention relates to a new and distinct cultivar of Heuchera hybrid and given the cultivar name of ‘Chinook’. Heuchera is in the family Saxifragaceae. Heuchera ‘Chinook’ originated as a tetraploid of Heuchera ‘Fireworks’ (U.S. Plant Pat. No. 11,376). Heuchera ‘Fireworks’ originated as a cross between Heuchera micrantha ‘Palace Purple’, (an unpatented plant) as the seed parent, and an unnamed coral flowered Heuchera sanguinea (an unpatented plant) as the pollen parent.

This new Heuchera is distinguished by:

1. Small habit

2. Large coral colored flowers on short flower stalks

3. Excellent vigor.

This new cultivar has been reproduced only by asexual propagation (division and tissue culture). Each of the progeny exhibits identical characteristics to the original plant. Asexual propagation by division and tissue culture using standard micropropagation techniques with terminal and lateral shoots, as done in Canby, Oreg., shows that the foregoing characteristics and distinctions come true to form and are established and transmitted through succeeding propagations. The present invention has not been evaluated under all possible environmental conditions. The phenotype may vary with variations in environment without a change in the genotype of the plant.

### BRIEF DESCRIPTION OF THE DRAWING

The drawing shows Heuchera ‘Chinook’ growing in a container in part sun in the garden in April in Canby, Oreg.

### DETAILED PLANT DESCRIPTION

The following is a detailed description of the new Heuchera cultivar based on observations of a two-year-old specimen grown in a container in the garden in April in Canby, Oreg. The color descriptions are all based on The Royal Horticultural Society Colour Chart.
Botanical classification: Heuchera hybrid
Variety denomination: ‘Chinook’
Parentage: Heuchera ‘Fireworks’ tetraploid
Plant:
   Form.—Basal rosette, herbaceous perennial.
   Hardiness.—USDA Zone 4 to 9.
   Size.—13 cm tall and 33 cm wide.
   Habit.—Clumping.
Leaf:
   Type.—Simple.
   Arrangement.—Rosette.
   Shape.—Ovate.
   Lobing/Division.—5 main lobes, with 1 to 2 shallow secondary lobes.
   Venation.—Palmate.
   Margins.—Crenate, incised and slightly undulate.
   Apex.—Mucronulate.
   Base.—Cordate, lobes overlapping at the base.
   Blade size.—6 cm long and 5.5 cm wide.
   Vestiture.—Glandular on both sides.
   Aspect.—Matte.
   Texture.—Thick, leathery.
   Petiole length.—Grows to 12.5 cm.
   Petiole vestiture.—Glandular hairs of various sizes.
   Petiole color.—Closest to Grayed Purple 177A.
   Leaf color.—Spring abaxial — Brown 200A with some green and purple tones Spring adaxial — Grayed Purple 187B Summer through winter abaxial — Between Green 147A and Brown 200A Summer through winter adaxial — Grayed Purple 187B.
Inflorescence:
   Size.—3 to 3.5 cm wide and 8 cm long.
   Type.—Thyrse.
   Number of flowers.—30 to 47 per thyrse.
   Number of thryse.—17.
   Peduncle.—With no prominent cauline leaves Height — 22 cm Diameter — 2.5 mm near base Color — Greyed Purple 187B Vestiture — Glandular.
   Branching.—0.
   Pedicel.—Length — Variable Vestiture — Glandular hairs Color — Grayed Purple 187B.
   Bloom period.—May in Canby, Oreg.
Flower bud:
   Size.—3.5 to 4 mm wide and 5 mm deep.
   Description.—Glandular puberulent, tear drop shaped.
   Color.—Deep Pink, Red 51B.
Flower:
   Type.—Perfect, zygomorphic.
   Shape.—Campanulate.
   Size.—11 mm deep and 10 mm wide.
   Petal count.—5, inconspicuous.
   Petal color.—Light pink, Red 49D.
   Petal shape.—Spatulate with a clawed base.
   Petal size.—3 mm long and 1.1 mm wide.
   Calyx.—5 lobes, divided ½ way to the base, with glandular hairs.
   Calyx size.—1 cm deep and wide.
   Calyx and flower color.—Coral on outside, overall Red 50C, with lighter edges on the lobes, Red 50D. Inside is Red 50D.
   Stigma, style color.—Yellow White 158D.
   Stamen number.—5.
   Anther color.—Yellow White 158D.
   Pollen.—None, plant male sterile.
   Fragrance.—None.
   Lastingness.—Each thryse blooms for about 2 weeks on the plant.
Fruit:
   Type.—Two-beaked ovoid capsule.
   Fertility.—Good.
   Color.—Brown 200D.
Seed:
   Shape.—Linear.
   Size.—2 mm. long.
   Color.—Black. RHS 202A.
Disease and pest tolerance: Excellent disease resistance to powdery mildew, the common problem of Heuchera. All Heuchera are susceptible to root weevils.

### COMPARISON TO SIMILAR HEUCHERAS

Compared to Heuchera ‘Fireworks’ (U.S. Plant Pat. No. 11,376), Heuchera ‘Chinook’ has much larger flowers on shorter flower stalks. The foliage of this new cultivar is thicker, darker and the habit is more compact.

I claim:

1. A new and distinct hybrid of Heuchera plant characterized by its large coral colored flowers on short stalks, dark slightly ruffled foliage, and a compact habit.
